SEQUENCE LISTING (44 sequences)

SEQ ID NO: 1 - DNA, 255 bp, Homo sapiens
  Human Parathyroid Hormone Coding Sequence (hPTH)
tctgtgagtgaaatacagcttatgcataacctgggaaaacatctgaactcgatggagagagtagaatggctgcgtaagaagctgcaggatgtgcacaattttgttgcccttggagctcctctagctcccagagatgctggttcccagaggccccgaaaaaaggaagacaatgtcttggttgagagccatgaaaaaagtcttggagaggcagacaaagctgatgtgaatgtattaactaaagctaaatcccagtga

SEQ ID NO: 2 - DNA, 252 bp, Bos sp.
  Bovine Parathyroid Hormone Coding Sequence (bPTH)
gctgtgagtgaaatacagtttatgcataacctgggcaaacatctgagctccatggaaagagtggaatggctgcggaaaaagctacaggatgtgcacaactttgttgcccttggagcttctatagcttacagagatggtagttcccagagacctcgaaaaaaggaagacaatgtcctggttgagagccatcagaaaagtcttggagaagcagacaaagctgatgtggatgtattaattaaagctaaaccccag

SEQ ID NO: 3 - DNA, 426 bp, Homo sapiens
  Human Parathyroid Hormone Related Protein Coding Sequence (PTHrP)
gctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggaggcattga

SEQ ID NO: 4 - DNA, 255 bp, Rattus sp.
  Rat Parathyroid Hormone Coding Sequence (rPTH)
gctgtcagtgaaatacagcttatgcacaacctgggcaaacacctggcctctgtggagaggatgcaatggctgagaaaaaagctgcaagatgtacacaattttgttagtcttggagtccaaatggctgccagagaaggcagttaccagaggcccaccaagaaggaggaaaatgtccttgttgatggcaattcaaaaagtcttggcgagggggacaaagctgatgtggatgtattagttaaggctaaatctcagtaa

SEQ ID NO: 5 - protein, 30 aa, Homo sapiens
  hPTH (1-31)
SVSEIQLMHNLGKHLNSMERVEWLRKLQDV

SEQ ID NO: 6 - protein, 40 aa, Homo sapiens
  PTHrP - (1-40)
AVSEHQLLHDKGKSIQDLRRRFFLHHLIAEIHTAEIRATS

SEQ ID NO: 7 - protein, 84 aa, Bos sp.
  bPTH
AVSEIQFMHNLGKHLSSMERVEWLRKKLQDVHNFVALGASIAYRDGSSQRPRKKEDNVLVESHQKSLGEADKADVDVLIKAKPQ

SEQ ID NO: 8 - protein, 22 aa, Artificial Sequence
  [Tyr63] hPTH (63-84)
YEKSLGEADKADVNVLTKAKSQ

SEQ ID NO: 9 - protein, 21 aa, Homo sapiens
  hPTH (64-84)
EKSLGEADKADVNVLTKAKSQ

SEQ ID NO: 10 - protein, 16 aa, Artificial Sequence
  [Tyr69] hPTH (69-84)
YADKADVNVLTKAKSQ

SEQ ID NO: 11 - protein, 15 aa, Homo sapiens
  hPTH (70-84)
ADKADVNVLTKAKSQ

SEQ ID NO: 12 - protein, 78 aa, Homo sapiens
  hPTH (7-84)
LMHNLGKHLNSMERVEWLRKKLQDVHNFVALGAPLAPRDAGSQRPRKKEDNVLVESHEKSLGEADKADVNVLTKAKSQ

SEQ ID NO: 13 - protein, 32 aa, Homo sapiens
  hPTHrP (1-31)
AVSEHQLLHDKGKSIQDLRRRFFLHHLIAEIH

SEQ ID NO: 14 - protein, 34 aa, Homo sapiens
  hPTH (1-34)
SVSEIQLMHNLGKHLNSMERVEWLRKKLQDVHNF

SEQ ID NO: 15 - protein, 84 aa, Homo sapiens
  hPTH
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 16 - protein, 84 aa, Rattus sp.
  rPTH
AVSEIQLMHNLGKHLASVERMQWLRKKLQDVHNFVSLGVQMAAREGSYQRPTKKEDNVLVDGNSKSLGEGDKADVDVLVKAKSQ

SEQ ID NO: 17 - protein, 34 aa, Bos sp.
  bPTH (1-34)
AVSEIQFMHNLGKHLSSMERVEWLRKKLQDVHNF

SEQ ID NO: 18 - protein, 34 aa, Homo sapiens; identical to SEQ ID NO: 14
  hPTH (1-34)

SEQ ID NO: 19 - protein, 34 aa, Artificial Sequence
  [Tyr1] hPTH (1-34)
YVSEIQLMHNLGKHLNSMERVEWLRKKLQDVHNF

SEQ ID NO: 20 - protein, 38 aa, Homo sapiens
  hPTH (1-38)
SVSEIQLMHNLGKHLNSMERVEWLRKKLQDVHNFVALG

SEQ ID NO: 21 - protein, 32 aa, Bos sp.
  bPTH (3-34)
SEIQFMHNLGKHLSSMERVEWLRKKLQDVHNF

SEQ ID NO: 22 - protein, 22 aa, Homo sapiens
  hPTH (13-34)
KHLNSMERVEWLRKKLQDVHNF

SEQ ID NO: 23 - protein, 22 aa, Artificial Sequence
  [Tyr27] hPTH (27-28)
YLQDVHNFVALGAPLAPRDAGS

SEQ ID NO: 24 - protein, 21 aa, Homo sapiens
  hPTH (28-48)
LQDVHNFVALGAPLAPRDAGS

SEQ ID NO: 25 - protein, 32 aa, Homo sapiens
  hPTH (53-84)
KKEDNVLVESHEKSLGEADKADVNVLTKAKSQ

SEQ ID NO: 26 - DNA, 22 bp, Artificial Sequence
  Oligonucleotide
agcggagacgatgcagcggaga

SEQ ID NO: 27 - DNA, 19 bp, Artificial Sequence
  Oligonucleotide
aagggaggcagctgagacg

SEQ ID NO: 28 - DNA, 21 bp, Artificial Sequence
  Oligonucleotide
gtccttggaaggtctctgctg

SEQ ID NO: 29 - DNA, 21 bp, Artificial Sequence
  Oligonucleotide
ttctagtgccactgcccattg

SEQ ID NO: 30 - DNA, 27 bp, Artificial Sequence
  Oligonucleotide
ctactaagctgtgtggatttctgcgat

SEQ ID NO: 31 - protein, 35 aa, Homo sapiens
  hPTHrP (1-34)
AVSEHQLLHDKGKSIQDLRRRFFLHHLIAEIHTAE

SEQ ID NO: 32 - protein, 141 aa, Homo sapiens
  hPTHrP (1-141)
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 33 - protein, 139 aa, Homo sapiens
  hPTHrP (1-139)
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 34 - protein, 209 aa, Homo sapiens
  hPTHrP (1-173)
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRTALLWGLKKKKENNRRTHHMQLMISLFKSPLLLL

SEQ ID NO: 35 - protein, 28 aa, Homo sapiens
  hPTH (7-34)
LMHNLGKHLNSMERVEWLRKKLQDVHNF

SEQ ID NO: 36 - protein, 29 aa, Homo sapiens
  hPTHrP (7-34)
LLHDKGKSIQDLRRRFFLHHLIAEIHTAE

SEQ ID NO: 37 - protein, 32 aa, Homo sapiens
  hPTH (5-36)
IQLMHNLGKHLNSMERVEWLRKKLQDVHNFVA

SEQ ID NO: 38 - protein, 32 aa, Homo sapiens
  hPTHrP (5-36)
HQLLHDKGKSIQDLRRRFFLHHLIAEIHTAEI

SEQ ID NO: 39 - protein, 30 aa, Homo sapiens
  hPTH (5-34)
IQLMHNLGKHLNSMERVEWLRKKLQDVHNF

SEQ ID NO: 40 - protein, 30 aa, Homo sapiens
  hPTHrP (5-34)
HQLLHDKGKSIQDLRRRFFLHHLIAEIHTA

SEQ ID NO: 41 - protein, 133 aa, Homo sapiens
  hPTHrP (7-139)
LLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 42 - protein, 135 aa, Homo sapiens
  hPTHrP (7-141)
LLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 43 - protein, 203 aa, Homo sapiens
  hPTHrP (7-173)
QQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRTALLWGLKKKKENNRRTHHMQLMISLFKSPLLLL

SEQ ID NO: 44 - protein, 44 aa, Homo sapiens
  hPTH (1-44)
SVSEIQLMHNLGKHLNSMERVEWLRKKLQDVHNFVALGAPLAPR

(19) United States
(12) Patent Application Publication
(10) Pub. No.: US 2004/0013719 A1
(43) Pub. Date: Jan. 22, 2004

(54) Regulation of cell proliferation and differentiation using topically applied nucleric acid molecules

(76) Inventor: Michael F. Holick, Sudbury, MA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/398,449

(22) PCT Filed: Oct. 5, 2001
(86) PCT No.: PCT/US01/31082

(51) Int. Cl. A61K 48/00; A61K 9/127; C12N 15/88

(52) U.S. Cl. 424/450; 514/44; 435/458

(57) ABSTRACT

Methods are disclosed for the regulation of cell differentiation and proliferation, e.g., for treating hyperproliferative skin disorder, such as psoriasis, and skin cancer for enhancing wound healing, for stimulating hair growth and inhibiting hair growth, by administration of nucleic acid molecules encoding parathyroid hormone (PTH), parathyroid related peptide (PTHrP), or fragment, analog or derivative thereof, and salts thereof, encapsulated by particular liposomes or incorporated into a porous biocompatable matrix.

### BACKGROUND OF THE INVENTION

1. Field of the Invention

This invention relates to the regulation of cell differentiation and proliferation, e.g., for treating hyperproliferative skin disorder, such as psoriasis, for enhancing wound healing, for stimulating hair growth, and inhibiting hair growth by topical administration of nucleic acid molecules encoding parathyroid hormone (PTH), parathyroid related peptide (PTHrP), or a fragment or analog thereof.

2. Related Art

U.S. Pat. Nos. 5,527,772, 5,840,690 and 6,066,618 describe methods of inhibiting proliferation and enhancing differentiation of mammalian cells, inducing proliferation of mammalian cells, enhancing wound healing, and stimulating hair growth using a peptide which has a 10% or greater homology to a region of human PTH or human PTHrP. Certain fragments and analogs (e.g. PTH (1-34), PTH (3-34) and PTHrP (1-34)) were found to act as agonists of PTH and PTHrP and inhibit proliferation and enhance differentiation of mammalian cells. Other fragments and analogs (e.g. PTH (7-34) and PTHrP (7-34) are antagonists of PTH and PTHrP were also found to enhance the proliferation of mammalian cells. The agonists are useful for treatment of hyperproliferative skin diseases such a psoriasis, actinic keratoses, and skin cancer and the antagonists are useful for wound healing, particularly wounds of the skin, enhancing or maintaining hair growth, particularly following chemotherapeutic treatment of a mammal, and stimulating epidermal regrowth. Methods of administration include oral, nasal, intravenous, topical, subcutaneous, parenteral and intraperitoneal administration. The peptides may be administered by subcutaneous pumps, patches, tapes, or by liposomal carriers.

A variety of PTH and PTHrP analogs and derivatives thereof have been made. See U.S. Pat. Nos. 4,086,196, 4,423,037, 4,771,124, 4,833,125, 4,968,669, 5,001,223, 5,087,562, 5,093,233, 5,116,952, 5,149,779, 5,171,670, 5,229,489, 5,317,010, 5,382,658, 5,393,869, 5,434,246, 5,527,772, 5,589,452, 5,807,823, 5,821,255, 5,840,690, 5,977,070, 6,025,467, 6,051,868, and 6,066,618; WO94/02510, WO00/23594, and WO00/31137; and EP 477,885. Methods for determining whether a particular analog is an agonist or antagonist of PTH and PTHrP are described in U.S. Pat. Nos. 5,527,772, 5,840,690 and 6,066,618.

Active vitamin D compounds are useful for treating hyperproliferative skin diseases and other conditions. A large number of such active vitamin D compounds are known. See U.S. Pat. Nos. 5,457,217, 5,414,098, 5,384,313, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

### SUMMARY OF THE INVENTION

The invention provides two important therapeutic methods one involving inhibition of cell proliferation and enhancement of skin cell differentiation (the agonist activity), which is useful in the treatment of psoriasis, ichthyosis, actinic keratoses, skin cancer, inhibiting hair growth or preventing hair regrowth. A second method involves enhancement of cell proliferation (the antagonist activity), which is useful in wound healing, stimulating epidermal regrowth and hair growth. In addition, the invention provides methods for enhancing wound healing and hair growth based on in vivo wound healing activity or in vitro or in vivo hair growth activity rather than strict agonist or antagonist activity in vitro.

The first method of the invention generally involves inhibiting proliferation and enhancing differentiation of mammalian skin cells by contacting the cell with a nucleic acid molecule encoding a peptide which is preferably at least 3, and more preferably at least 8, amino acids long and has 10% or greater (more preferably, 50% or greater, and most preferably 75% or 10 greater) sequence identity with a region (preferably within the amino-terninal 34 amino acid region) of human PTH or human PTHrP and, when expressed, is capable of inhibiting proliferation or enhancing the differentiation in vitro of cultured human keratinocytes; or in vivo in mouse skin by inhibiting skin cell proliferation or hair cycle progression or hair growth. In preferred embodiments of this method, the peptide encoded by the nucleic acid molecule is hPTH (1-84), hPTH (1-34), hPTHrP (1-31), hPTHrP (1-40), hPTH (1-44), hPTH (1-36), hPTH (1-38), hPTH (1-31), hPTH (3-34), hTHrP (1-34), hPTHrP (1-141), hPTHrP (1-139) or hPTHrP (1-173). This method has particular application in the treatment of hyperproliferative skin disorders such as psoriasis. The method may also be useful in the treatment of certain preskin cancers and skin cancers, by the inhibition of cancer cell proliferation and by the induction of differentiation and inhibition of hair growth or preventing hair growth and acne.

The second method of the invention generally involves enhancing proliferation of mammalian skin cells by contacting the skin cells with a nucleic acid molecule encoding a peptide which is preferably at least 3, and more preferably at least 8, amino acids long and has 10% or greater (more preferably, 50% or greater, and most preferably 75% or greater) sequence identity with a region (preferably within the amino-terminal 34 amino acid region) of hPTH or hPTHrP and, when expressed, is capable of blocking the differentiation or the inhibition of proliferation in vitro of cultured human keratinocytes by PTH (1-34) or 1,25(OH)₂D₃ or PTHrP (1-34); or in vivo in mouse skin by stimulating skin cell proliferation or accelerating hair cycle progression or stimulating hair growth. In a preferred embodiment of this method, the peptide encoded by the nucleic acid molecule is PTH (7-34), PTH (7-84), hPTH (5-34), hPTHrP (7-34), hPTHrP (5-34), hPTHrP (7-141), hPTHrP (7-134), or hPTHrP (7-173). In a related method of the invention, proliferation of mammalian skin cells, e.g., during wound healing, is enhanced by contacting the cell or wound with nucleic acid molecule encoding a peptide which is preferably at least 3, and more preferably at least 8, amino acids long and has 10% or greater (more preferably, 50% or greater, and most preferably, 75% or greater) sequence identity with a region (preferably, within the amino-terminal 34 amino acid region) of hPTH or hPTHrP, and, when expressed, is capable of enhancing wound healing in an in vivo skin punch assay. In preferred embodiments of this method, the peptide encoded by the nucleic acid molecule is hPTH (1-84), hPTH (1-34), hPTH (7-34), hPTH (5-34), hPTH (5-36), hPTH (1-31), hPTHrP (1-34), hPTHrP (1-135), hPTHrP (1-141), hPTHrP (1-173) or hPTHrP (7-34). These related methods have particular application in the enhancement of wound healing and also have applications in the promotion of skin growth in patients with burns or skin ulcerations as well as in the stimulation of epidermal regrowth in people who have decreased epidermal cell proliferation due to aging.

Hair growth is stimulated by administering to a mammal a nucleic acid molecule encoding a peptide which is preferably at least 3, and more preferably at least 8, amino acids long and has 10% or greater (more preferably, 50% or greater, and most preferably, 75% or greater) sequence identity with a region (preferably, within the amino-terminal 34 amino acid region) of hPTH or hPTHrP, and, when expressed, is capable of stimulating hair growth in vitro or in vivo. In preferred embodiments of this method, the peptide encoded by the nucleic acid molecule is hPTH (7-34), hPTH (7-84), hPTHrP (7-134), hPTHrP (7-141), hPTHrP (7-173), hPTH (5-34), hPTHrP (7-34) or hPTH (5-36).

The nucleic acid molecules are administered as part of a pharmaceutical composition comprising a pharmaceutically acceptable carrier. In a preferred embodiment, the carrier is a liposome or gel. In another preferred embodiment the nucleic acid molecules are contained within a porous biocompatable matrix.

The invention also relates to a method of inhibiting proliferation or enhancing differentiation of a skin or hair cell of a mammal, comprising administering to the mammal in need thereof a proliferation-inhibiting or differentiation-enhancing amount of a nucleic acid molecule of the invention and an active vitamin D compound, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth. The invention also relates to a composition comprising a nucleic acid molecule of the invention encapsulated within a liposome.

The invention also relates to a composition comprising a proliferation-inhibiting or differentiation-enhancing amount of a nucleic acid molecule of the invention and an active vitamin D compound, optionally encapsulated within a liposome.

Other features and advantages of the invention will be apparent from the following description of the preferred embodiments thereof, and from the claims.

### BRIEF DESCRIPTION OF THE FIGURES

FIG. 1 depicts a bar graph showing the effect of transfecting PTHrP (1-141) and PTHrP (1-173) genes into cultured keratinocytes on ³H-thymidine incorporation. Bar 1 represents the empty vector, Bar 2 represents the PTHrP gene (1-141) transfected into cultured human keratinocytes, Bar 3 represents the PTHrP gene (1 -173) transfected into cultured human keratinocytes.

FIGS. 2A-2C depict schematic representations of the cDNA structure of the PTHrP (1-139), PTHrP (1-141) and PTHrP (1-173) genes.

FIG. 3 depicts a schematic representation of the pACCMV.pLpa adenoviral expression vector.

FIG. 4 depicts the sequence of SEQ ID NO: 1.

FIG. 5 depicts the sequence of SEQ ID NO: 2.

FIG. 6 depicts the sequence of SEQ ID NO: 3.

FIG. 7 depicts the sequence of SEQ ID NO: 4.

FIG. 8 depicts the sequence of SEQ ID NO: 5.

FIG. 9 depicts the sequence of SEQ ID NO: 6.

FIG. 10 depicts the sequence of SEQ ID NO: 7.

FIG. 11 depicts the sequence of SEQ ID NO: 8.

FIG. 12 depicts the sequence of SEQ ID NO: 9.

FIG. 13 depicts the sequence of SEQ ID NO: 10.

FIG. 14 depicts the sequence of SEQ ID NO: 11.

FIG. 15 depicts the sequence of SEQ ID NO: 12.

FIG. 16 depicts the sequence of SEQ ID NO: 13.

FIG. 17 depicts the sequence of SEQ ID NO: 14.

FIG. 18 depicts the sequence of SEQ ID NO: 15.

FIG. 19 depicts the sequence of SEQ ID NO: 16.

FIG. 20 depicts the sequence of SEQ ID NO: 17.

FIG. 21 depicts the sequence of SEQ ID NO: 18.

FIG. 22 depicts the sequence of SEQ ID NO: 19.

FIG. 23 depicts the sequence of SEQ ID NO: 20.

FIG. 24 depicts the sequence of SEQ ID NO: 21.

FIG. 24 depicts the sequence of SEQ ID NO: 22.

FIG. 26 depicts the sequence of SEQ ID NO: 23.

FIG. 27 depicts the sequence of SEQ ID NO: 24.

FIG. 28 depicts the sequence of SEQ ID NO: 25.

FIG. 29 depicts the sequence of SEQ ID NO: 26.

FIG. 30 depicts the sequence of SEQ ID NO: 27.

FIG. 31 depicts the sequence of SEQ ID NO: 28.

FIG. 32 depicts the sequence of SEQ ID NO: 29.

FIG. 33 depicts the sequence of SEQ ID NO: 30.

FIG. 34 depicts the sequence of SEQ ID NO: 31.

FIG. 35 depicts the sequence of SEQ ID NO: 32.

FIG. 36 depicts the sequence of SEQ ID NO: 33.

FIG. 37 depicts the sequence of SEQ ID NO: 34.

FIG. 38 depicts the sequence of SEQ ID NO: 35.

FIG. 39 depicts the sequence of SEQ ID NO: 36.

FIG. 40 depicts the sequence of SEQ ID NO: 37.

FIG. 41 depicts the sequence of SEQ ID NO: 38.

FIG. 42 depicts the sequence of SEQ ID NO: 39.

FIG. 43 depicts the sequence of SEQ ID NO: 40.

FIG. 44 depicts the sequence of SEQ ID NO: 41.

FIG. 45 depicts the sequence of SEQ ID NO: 42.

FIG. 46 depicts the sequence of SEQ ID NO: 43.

FIG. 47 depicts the sequence of SEQ ID NO: 44.

### DESCRIPTION OF THE PREFERRED EMBODIMENTS

Nucleic Acid Molecules of the Invention

The invention relates to the regulation of cell differentiation and proliferation by administration of nucleic acid molecules encoding parathyroid hormone (PTH), parathyroid hormone related protein (PTHrP), or a fragment or analog thereof. Particular nucleic acid molecules which can be used include those which encode the following peptides:

hPTH (1-84), encoded by nucleotides 1-252 of the nucleic acid molecule of SEQ ID NO: 1 (Kimura, T. et al., BBRC 11:.493 (1983); Fairwell, T. et al., Biochemistry 22:691 (1983)).

hPTH (1-31), encoded by nucleotides 1-93 of the nucleic acid molecule of SEQ ID NO: 1.

hPTH (1-34), encoded by nucleotides 1-102 of the nucleic acid molecule of SEQ ID NO: 1.

HPTH (1-36), encoded by nucleotides 1-108 of the nucleic acid molecule of SEQ ID NO: 1.

hPTH (1-38), encoded by nucleotides 1-114 of the nucleic acid molecule of SEQ ID NO: 1 (Heech, R. D. et al., Horm. Metab. Res. 16:556 (1984)).

hPTH (1-44), encoded by nucleotides 1-132 of the nucleic acid molecule of SEQ ID NO: 1 (Kimura T. et al., Biopolymers 20:1823 (1981)).

hPTH (5-36), encoded by nucleotides 13-108 of the nucleic acid molecule of SEQ ID NO: 1.

HPTH (7-34), encoded by nucleotides 19-102 of the nucleic acid molecule of SEQ ID NO: 1.

hPTH (13-34), encoded by nucleotides 40-102 of the nucleic acid molecule of SEQ ID NO: 1.

hPTH (28-48), encoded by nucleotides 82-144 of the nucleic acid molecule of SEQ ID NO: 1 (Rosenblatt, M. et al., Biochemistry 16:2811 (1977)).

HPTH (7-84), encoded by nucleotides 19-252 of the nucleic acid molecule of SEQ ID NO: 1.

hPTH (53-84), encoded by nucleotides 157-252 of the nucleic acid molecule of SEQ ID NO: 1 (Rosenblatt, M. et al., Endocrinology 103:976 (1978)).

hPTH (64-84), encoded by nucleotides 190-252 of the nucleic acid molecule of SEQ ID NO: 1.

hPTH (70-84), encoded by nucleotides 208-252 of the nucleic acid molecule of SEQ ID NO: 1.

[Tyr¹]-hPTH (1-34), encoded by nucleotides 1-102 of the nucleic acid molecule of SEQ ID NO: 1, wherein the adenosine at position 2 is mutated to a cytosine.

[Tyr²⁷]-hPTH (27-48), encoded by nucleotides 79-144 of the nucleic acid molecule of SEQ ID NO: 1, wherein the adenosine at position 79 and the guanosine at position 81 are both mutated to a thymidine.

[Tyr⁶³]-hPTH (63-84), encoded by nucleotides 187-252 of the nucleic acid molecule of SEQ ID NO: 1, wherein the cytosine at position 187 is mutated to a thymidine.

[Tyr⁶⁹]-hPTH (69-84), encoded by nucleotides 205-255 of the nucleic acid molecule of SEQ ID NO: 1, wherein the guanosine at position 205 is mutated to a thymidine, and the guanosine at position 207 is mutated to either a thymidine or a cytosine.

PTH, Bovine (bPTH) (1-84), encoded by nucleotides 1-252 of the nucleic acid molecule of SEQ ID NO: 2.

bPTH (1-34), encoded by nucleotides 1-102 of the nucleic acid molecule of SEQ ID NO: 2 (Tregear, G. W. et al., Biochemistry 16:2817 (1977)).

bPTH (3-34), encoded by nucleotides 7-102 of the nucleic acid molecule of SEQ ID NO: 2 (Lowrik, C. et al., Cell Calcium 6:311 (1985)).

PTHrP (1-31), encoded by nucleotides 1-93 of the nucleic acid molecule of SEQ ID NO: 3.

PTHrP (1-40), encoded by nucleotides 1-120 of the nucleic acid molecule of SEQ ID NO: 3.

PTHrP (5-36), encoded by nucleotides 13-108 of the nucleic acid molecule of SEQ ID NO: 3.

PTHrP (7-34), encoded by nucleotides 19-102 of the nucleic acid molecule of SEQ ID NO: 3.

PTHrP (7-139), encoded by nucleotides 19-417 of the nucleic acid molecule of SEQ ID NO: 3.

PTHrP (7-141), encoded by nucleotides 19423 of the nucleic acid molecule of SEQ ID NO: 3.

PTHrP (7-173), encoded by nucleotides 19-519 of the nucleic acid molecule of SEQ ID NO: 3.

Rat PTH (rPTH) (1-84), encoded by nucleotides 1-252 of the nucleic acid molecule of SEQ ID NO: 4 (Heinrich, G. et al., J. Biol. Chem. 25:3320 (1984)).

In addition, nucleic acid molecules which encode the peptides and peptide derivatives disclosed in the following documents can also be used: U.S. Pat. Nos. 4,086,196, 4,423,037, 4,771,124, 4,833,125, 4,968,669, 5,001,223, 5,087,562, 5,093,233, 5,116,952, 5,149,779, 5,171,670, 5,229,489, 5,317,010, 5,382,658, 5,393,869, 5,434,246, 5,527,772, 5,589,452, 5,807,823, 5,821,255, 5,840,690, 5,977,070, 6,025,467, 6,051,868, and 6,066,618; WO94/02510, WO00/23594, and WO00/31137; and EP 477,885.

A typical design for constructing the PTH (7-34), (7-84), (7-141), and PTHrP (7-34), (7-139), and (7-173) fragment cDNAs is to place a ATG start codon upstream of the initial peptide codon of the individual fragments and to introduce a stop codon downstream of the final peptide codon of the individual fragments. Also, an endogenous peptide cleavage site will be introduces between the ATG start codon and the initial peptide codon of the individual fragments to avoid unwanted amino acids being introduced into the constructs.

When selecting a candidate nucleic acid molecule for a method of this invention, a preferred first step is to choose a nucleic acid molecule encoding a peptide which includes a fragment which has at least 10%, and more preferably 50% or greater, sequence identity with an 8 or greater amino acid long fragment within the amino terminal 34 amino acid region of hPTH or hPTHrP. The term “sequence identity” refers to a measure of the identity of nucleotide sequences or amino acid sequences. In general, the sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(i):387 (1984)), BLASTP, BLASTN, FASTA (Atschul, S. F., et al., J Molec Biol 215:403 (1990)).

Therefore, as used herein, the term “identity” represents a comparison between a test and reference sequence. More specifically, reference test sequence is defined as any test sequence that is 10% or more identical to a reference sequence. As used herein, the term at least 10% identical to refers to percent identities from 10 to 99.99 relative to the reference sequence. Identity at a level of 10% or more is indicative of the fact that, assuming for exemplification purposes a test and reference sequence length of 100 amino acids, that no more than 90% (i.e., 90 out of 100) of the amino acids in the test sequence differ from that of the reference sequence. Such differences may be represented as point mutations randomly distributed over the entire length of the nucleotide or amino acid sequence of the invention or they may be clustered in one or more locations of varying length up to the maximum allowable amino acid difference. Differences are defined as nucleotide or amino acid substitutions, or deletions.

Because of the high degree of homology among human PTH and PTH of other species, PTH peptides encoded by nucleic acids from non-human as well as human sources can be used. Similarly, human PTHrP (1-139), (1-141) and (1-173) have a high degree of homology with PTHrP of other species; therefore, nucleic acids from non-human as well as human sources can be used in the methods of the invention involving PTHrP.

Candidate nucleic acid molecules may be tested for suitability as inhibitors of cell proliferation and enhancers of differentiation using cultured human keratinocytes, similar to the method for testing peptides described in U.S. Pat. Nos. 5,527,772, 5,840,690 and 6,066,618. Briefly, those nucleic acid molecules encoding peptides which inhibit proliferation and induce differentiation in cultured keratinocytes are those potentially useful as therapeutic agents in treating disorders, e.g., psoriasis and cancer, where suppression of cell proliferation is desired. Candidate nucleic acid molecules may be tested for suitability as enhancers of cell proliferation using cultured human keratinocytes or ice vivo mouse model. Those peptides encoded by the nucleic acid molecules which block the effect of agonist peptides or 1,25(OH)₂D₃ on cultured keratinocyte proliferation are those potentially useful as therapeutic agents in treating disorders, e.g., wounds, burns, or skin ulcerations, where maintenance or stimulating of cell proliferation is desired.

Candidate nucleic acid molecules may be tested for their ability to enhance wound healing by carrying out a skin punch biopsy test, as described in U.S. Pat. Nos. 5,527,772, 5,840,690 and 6,066,618.

Candidate peptides may be tested for suitability as stimulators of hair growth using an in vitro hair growth assay, as described in U.S. Pat. Nos. 5,527,772, 5,840,690 and 6,066,618. Those peptides encoded by the nucleic acid molecules which stimulate hair growth in vitro are those potentially useful for the stimulation of hair growth in vivo, e.g., for the stimulation or maintenance of hair growth during or following chemotherapy or to treat a form of alopecia, e.g., male and female pattern baldness.

Alternatively, in vivo assays may be carried out as described herein and similar to those described in Schilli, M. B. et al., J. Invest. Dermatol. 108:928-932 (1997); Holick, M. F., et al., Proc. Natl. Acad Sci. 91:8014-8016 (1994); Paus, R. and Cotsarelis, G., N. Engl. J. Med 341: 491-497 (1999); Paus, R., et al. Laboratory Invest. 60: 365-369 (1989) and U.S. Pat. App. No. 60/213,247.

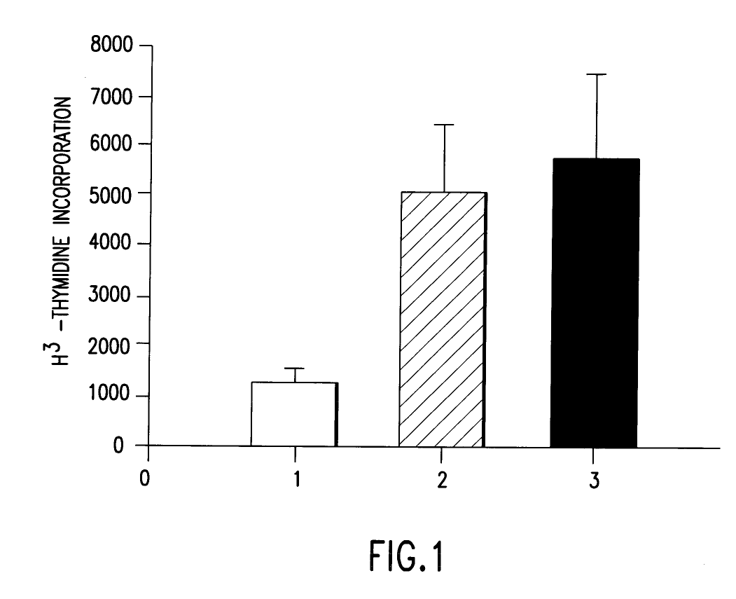

Care should be taken when determining the correct nucleic acid molecule for use in the invention. Experiments have shown that when normal cultured human keratinocytes are transfected with plasmids containing PTHrP (1-141) or PTHrP (1-173) an unexpected enhancement of cell growth is seen, as measured by ³H-thymidine incorporation into epidermal DNA (FIG. 1). These results are attributed to proteolysis of the full-length peptide. For this reason, all candidate nucleic acid molecules should be tested for the expected activity before use.

Gene Therapy

In this preferred embodiment of the invention, a nucleic acid molecule encoding a peptide with desired activity is incorporated into a polynucleotide construct suitable for introducing the nucleic acid molecule into cells of the animal to be treated, to form a transfection vector. The transfection vector is then introduced into selected target tissues of the cells of the animal in vivo using any of a variety of methods known to those skilled in the art. Alternatively, naked DNA may be transfected into the cells, with or without cationic lipids.

Techniques for the construction of transfection vectors containing inserts of desired nucleic acid sequences are well-known in the art, and are generally described in “Working Toward Human Gene Therapy,” Chapter 28 in Recombinant DNA, 2nd Ed., Watson, J. D. et al. (eds.), Scientific American Books: New York (1992), pp. 567-581, or Sambrook et al., Molecular Cloning: A Laboratory Manual, Cold Spring Harbor, New York (1989).

Gene therapy approaches that may be used to deliver a nucleic acid molecule include injection of plasmid DNA (Horton, H. M., et al., Proc. Natl. Acad. Sci. USA 96(4):1553-1558 (1999)); transduction using adenoviral vectors (Waugh, J. M., et al., Proc. Natl. Acad. Sci. USA 96(3):1065-1070 (1999)); transduction using retrovial vectors (Axelrod, J. H., et al., Proc. Natl. Acad. Sci. USA 87:5173-5177 (1990); Drumm, M. L., et al., Cell 62:1227-1233 (1990); Krueger, G. G., et al., J. Invest. Dermatol. 112:233-239 (1999); Palmer, T. D., et al., Blood 73:438-445 (1989); and Rosenberg, S. A., et al., N. Eng. J Med. 323:570-578 (1990)); and gene transfer using liposomes (Mason, C. A. E., et al., Nature Medicine 5(2):176-182 (1999)). In addition, general methods for construction of gene therapy vectors and the introduction of such vectors into a mammal for therapeutic purposes may be obtained in the above-referenced publications, the disclosures of which are specifically incorporated herein by reference in their entirety. In one such general method, vectors containing nucleic acid sequences of the present invention are directly introduced into the cells or tissues of the mammal to be treated, preferably by topical application. Such an approach is generally referred to as “in vivo” gene therapy.

Alternatively, cells or tissues may be removed from the mammal to be treated and placed into culture according to methods that are well-known to one of ordinary skill in the art. Transfection vectors or naked DNA containing the genes for desired peptides may then be introduced into these cells or tissues by any of the methods described generally above for introducing isolated polynucleotides into a cell or tissue. After a sufficient amount of time to allow incorporation of the inserted DNA, the cells or tissues may then be re-inserted into the mammal to be treated. Since introduction of the nucleic acid molecule encoding the peptide is performed outside of the body of the mammal, this approach is generally referred to as “ex vivo” gene therapy. See U.S. Pat. No. 5,399,346. Gene transfer through transfection of cells ex vivo can be performed by a variety of methods, including, for example, calcium phosphate precipitation, diethylaminoethyl dextran, electroporation, lipofection, or viral infection. Such methods are well known in the art (see, for example, Sambrook et al.).

For both in vivo and ex vivo gene therapy, the nucleic acid molecule encoding the desired peptide of the invention may be operatively linked to a regulatory DNA sequence, or “promoter,” to form a genetic construct as described above. This construct, containing both the promoter and the nucleic acid molecule encoding the peptide, may be subcloned into a suitable vector such as a plasmid, adenovirus vector, retrovirus vector, or the like, and introduced into the animal to be treated in an in vivo gene therapy approach, or into the cells or tissues of the mammal in an ex vivo approach.

Alternatively, the nucleic acid molecule of the invention may be operatively linked to a heterologous regulatory DNA sequence, or promoter, to form a genetic construct as described above. The heterologous regulatory sequence may be tissue specific. The vector containing the genetic construct is then directly introduced into the animal to be treated or into the cells or tissues of the animal, as described.

The term “operably linked”, as used herein, denotes a relationship between a regulatory region (typically a promoter element, but may include an enhancer element) and the gene, whereby the transcription of the gene is under the control of the regulatory region.

The term “heterologous” means a DNA sequence not found in the native genome. That is, two nucleic acid elements are said to be “heterologous” if the elements are derived from two different genes, or alternatively, two different species. Thus, “heterologous DNA regulatory sequence” indicates that the regulatory sequence is not naturally ligated to the nucleic acid molecule selected for use in the invention.

The term “promoter” is used according to its art-recognized meaning. It is intended to mean the DNA region, usually upstream to the coding sequence of a gene, which binds RNA polymerase and directs the enzyme to the correct transcriptional start site.

In general, a promoter may be functional in a variety of tissue types and in several different species of organisms, or its function may be restricted to a particular species and/or a particular tissue. Further, a promoter may be constitutively active, or it may be selectively activated by certain substances (e.g., a tissue-specific factor), under certain conditions (e.g., in the presence of an enhancer element, if present, in the genetic construct containing the promoter), or during certain developmental stages of the organism (e.g., active in fetus, silent in adult).

Promoters useful in the practice of the present invention are preferably “tissue-specific”—that is, they are capable of driving transcription of a gene in one tissue while remaining largely “silent” in other tissue types. Examples of tissue-specific promoters in the skin are the Keratin promoter (Vassar et al., Proc. Natl. Acad. Sci. U.S.A. 86:8565 (1989)), the POMC promoter (Deen et al. Mol. Biol. Evol. 9:483 (1992)), the alpha-actin promoter (Shani, Mol. Cell. Biol., 6:2624 (1986)), the elastase-q promoter (Swift et al., Cell 28:639 (1984)), the tyrosine hydroxylase promoter (Kim, L. S., et al., J. Biol. Chem 268:15689 (1993); Kaneda, N., et al., Neuron 6:583 (1991)), the dopamine beta-hydroxylase promoter (Mercer E. H., et al., Neuron 7:703 (1991); Hcyle, G. W., et al., J. Neurosci. 14:2455 (1994)), the tryptophan hydroxylase promoter (Boularand, S., et al., J. Biol. Chem 270:3757 (1995); Stoll, J. and Goldman, D., J. Neurosci. Res. 28:457 (1991)) and the parathyroid hormone-related peptide promoter (Campos, R. V., et al., Mol. Rnfovtinol. 6:1642). For additional examples of tissue-specific promoters, see U.S. Pat. Nos. 5,834,306 and 5,416,027, and references cited therein.

In addition to a promoter, the genetic construct may also contain other genetic control elements, such as enhancers, repressible sequences, and silencers, which may be used to regulate replication of the vector in the target cell. The only requirement is that the genetic element be activated, derepressed, enhanced, or otherwise genetically regulated by factors in the host cell and, with respect to methods of treatment, not in the non-target cell.

An “element,” when used in the context of nucleic acid constructs, refers to a region of the construct or a nucleic acid fragment having a defined function.

For example, an enhancer element, as used herein, is a region of DNA that, when associated with inserted nucleic acid molecule, operably linked to a promoter, enhances the transcription of that gene.

The term “enhancer” is used according to its art-recognized meaning. It is intended to mean a sequence found in eukaryotes which can increase transcription from a gene when located (in either orientation) up to several kilobases from the gene being studied. These sequences usually act as enhancers when on the 5′ side (upstream) of the gene in question. However, some enhancers are active when placed on the 3′ side (downstream) of the gene. In some cases, enhancer elements can activate transcription from a gene with no (known) promoter.

Preferred enhancers include the DF3 breast cancer-specific enhancer and enhancers from viruses and the steroid receptor family. Other preferred transcriptional regulatory sequences include NF1, SP1, AP1, and FOS/JUN.

Any of a variety of methods known to those skilled in the art may be used to introduce transfection vectors of the present invention into selected target tissue cells. Such methods include, for example, viral-mediated gene transfer using retroviruses, adeno-associated virus (AAV), herpes virus, vaccinia virus, or RNA viruses (e.g., Grunhaus and Horowitz, Semin. Virol 3:237-252 (1992); Herz and Gerard, Proc. Nat. Acad. Sci. USA 90:2812-2816 (1993); and Rosenfeld et al., Cell 68:143-155 (1992)); liposome-mediated gene transfer (Morishita et al., J. Clin. Invest. 91:2580 (1993); Feigner et al., U.S. Pat. Nos. 5,703,055 (1997) and 5,858,784 (1999)); injection of naked DNA directly into a target tissue (e.g., Feigner et al., U.S. Pat. No. 5,589,466 (1996); Wolff et al., U.S. Pat. No. 5,693,622 (1997)); and receptor-mediated gene transfer (Wu and Wu, Biochemistry 27:887-892 (1988); Wagner et al., PNAS USA 87:3410-3414 (1990); Curiel et al., U.S. Pat. No. 5,547,932 (1996); and Beug et al., U.S. Pat. No. 5,354,844 (1994)).

In any of these methods, where a vector may be targeted to selectively transfect a specific population of cells, it will be understood that in addition to local administration (such as may be achieved by injection into the target tissue), the vector may be administered systemically (e.g., intravenously) in a biologically-compatible solution or pharmaceutically acceptable delivery vehicle. Vector constructs administered in this way may selectively infect the target tissue. According to the present invention, the presence of a target tissue-specific promoter on the construct provides an independent means of restricting expression of the therapeutic gene.

Nucleic acid molecules encoding peptides which block antiproliferative compounds can also be useful in conjunction with chemotherapeutic agents in the treatment of skin cancer; many chemotherapeutic agents are effective only against dividing cells, and the blocking peptides can have the effect of inducing division of otherwise dormant cells, rendering them vulnerable to the chemotherapy. Nucleic acids encoding blocking peptides can also be useful in promoting growth of new cells, e.g., skin cells, in topical skin creams. Differentiation-inducing peptides can be used as immunostimulants, by inducing maturation of monocytes and lymphocytes bearing PTH receptors, while blocking peptides can be used to inhibit lymphocyte maturation, and thus can be used to treat conditions, e.g., autoimmune diseases such as juvenile diabetes, rheumatoid arthritis, and allograft rejection, where mature lymphocytes are a causative agent. The nucleic acid molecules of the invention can be admixed with a pharmacologically inert topical carrier such as one comprising a gel, an ointment or a cream, including such carriers as water, glycerol, alcohol, propylene glycol, fatty alcohol, triglycerides, fatty acid ester or mineral oils. Other possible carriers are liquid petrolatum, isopropylpalmitate, polyethylene glycol ethanol 95%, polyoxyethylene monolaurate 5% in water, sodium lauryl sulfate 5% in water, and the like. Materials such as antioxidants, humectants, viscosity stabilizers and the like may be added, if necessary. Nucleic acid molecules can be incorporated into liposomes using methods outlined in U.S. Pat. No. 5,260,065.

The nucleic acid molecules can be incorporated into a collagenous biocompatable matrix similar to the methods utilized in Fang et al., Proc. Nat. Acad. Sc. USA. 93:5753 (1996) and U.S. Pat. No. 5,962,427. The types of matrices that may be used in the practice of the invention is virtually limitless and may include both biological and synthetic matrices. The matrices may be biodegradable or non-biodegradable. The matrices may take the form of sponges, implants, tubes, telfa pads, band-aids, bandages, pads, lyophylized components, gels, patches, powders or nanoparicles. Particular examples of such matrices include porous or collagenous materials (e.g. type II collagen), hydroxyapatite, bioglass, aluminates, bioceramic materials, purified proteins or extracellular matrix compositions as well as metals such as titanium.

The nucleic acid molecules can be provided in the form of pharmaceutically acceptable salts. Examples of preferred salts are those of therapeutically acceptable organic acids, e.g., acetic, lactic, maleic, citric, malic, ascorbic, succinic, benzoic, salicylic, methanesulfonic, toluenesulfonic, or pamoic acid, as well as polymeric acids such as tannic acid or carboxymethyl cellulose, and salts with inorganic acids such as hydrohalic acids, e.g, hydrochloric acid, sulfuric acid, or phsophoric acid.

Dosage will be dependent upon the age, health, and weight of the recipient; kind of concurrent treatment, if any; frequency of treatment; and the nature of the effect desired. Generally, daily dosage may be 0.001 to 500 μg/kg. The topical dosage may be from 0.01 to 100 μg/cm². The liposomal gel, ointment or cream formulations may be applied by one or more applications per day.

The invention also relates to compositions comprising a nucleic acid molecule of the invention, an active vitamin D compound and a pharmaceutical carrier, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair growth. A large number of active vitamin D compounds are known which can be used in the practice of the present invention. See U.S. Pat. Nos. 5,457,217, 5,414,098, 5,384,313, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791. A preferred active vitamin D compound is calcipotriene. In this embodiment, any conventional liposome may be used including the liposomes described in U.S. Pat. Nos. 4,235,871, 4,241,046, 4,247,411, 4,356,167, 4,377,567, 4,544,545, 4,551,288, 4,610,868, 4,731,210, 4,744,989, 4,772,471, 4,897,308, 4,917,951, 5,021,200, 5,032,457, and 5,260,065.

The invention relates as well to a method of inhibiting proliferation or enhancing differentiation of a skin or hair cell of a mammal, comprising administering to the mammal in need thereof a proliferation-inhibiting or differentiation-enhancing amount of a nucleic acid molecule of the invention and an active vitamin D compound, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth. In this embodiment, the nucleic acid molecule encoding the peptide and the active vitamin D compound may be administered as part of single or separate pharmaceutical compositions. Either one or both of the nucleic acid molecules and active vitamin D compound may be administered topically or parenterally. In a preferred embodiment, the nucleic acid molecule is administered first followed by the active vitamin D compound.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE1

Mini-Gene Construction

PTHrP gene: PTHrP gene expresses three isoform peptides: PTHrP 1-139, PTHrP1-141 and PTHrP 1-173. The gene splicing happens between exon 4 to exon 6. The 5′-flanking regions share common nucleotide sequences, including precursor peptide. PTHrP mini-genes were made based on the nucleotide sequences of the human PTHrP/PLP gene, (Yasuda et al. J. Biol. Chem. 264:7720 (1989)) by using the PCR technique. The interested gene fragments were constructed into pCR3.1eukaryotic expression vector. The forward primer for PTHrP (1-139), PTHrP (1-141), PTHrP (1-173) and PTHrP (1-34) is 5′-AGCGGAGACGATGCAGCGGAGA-3′ (SEQ ID NO: 26), reverse primer for PTHrP (1-139) is 5′-AAGGGAGGCAGCTGAGACG-3′ (SEQ ID NO: 27), for PTHrP (1-141) is 5′-GTCCTTGGAAGGTCTCTGCTG-3′ (SEQ ID NO: 28), for PTHrP (1-173) is 5′-TTCTAGTGCCACTGCCCATTG-3′ (SEQ ID NO: 29) and for PTHrP (1-34) is 5′-CTACTAAGCTGTGTGGATTTCTGCGAT-3′ (SEQ ID NO: 30). PCR was performed at 94° C. for 3 min initial denaturing, then followed by denaturing for 30 seconds at 94° C., annealing for 30 seconds at 60° C. and extension for 1 min at 72° C., total 30 cycles, additional extension for 10 min at 72° C.

Adenovirus Construction of PTHrP

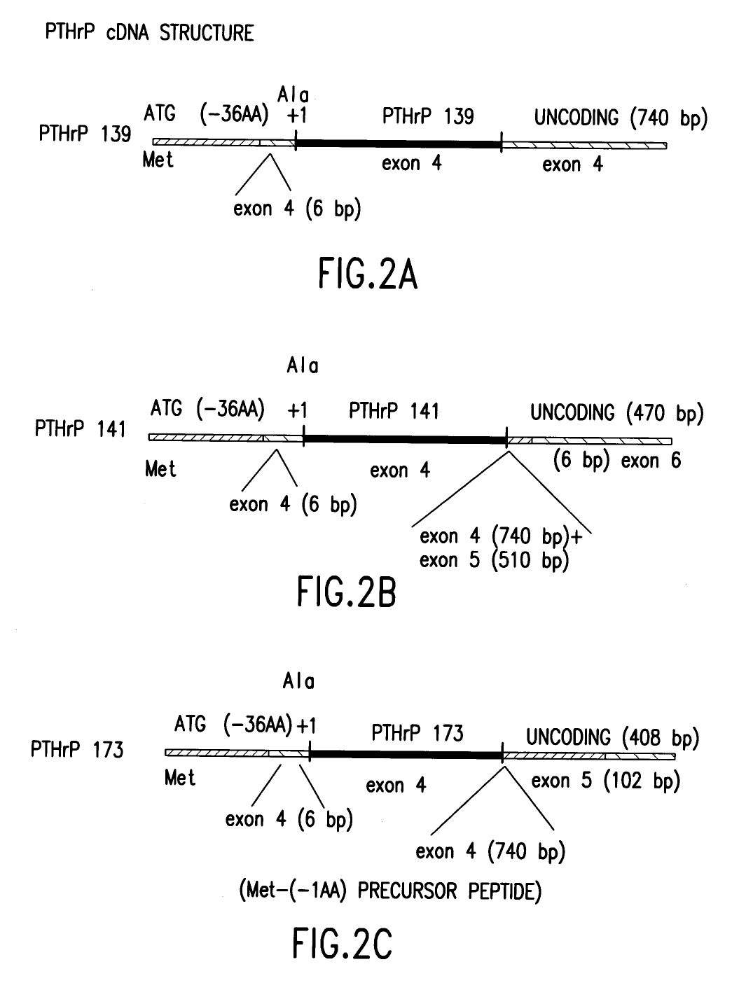

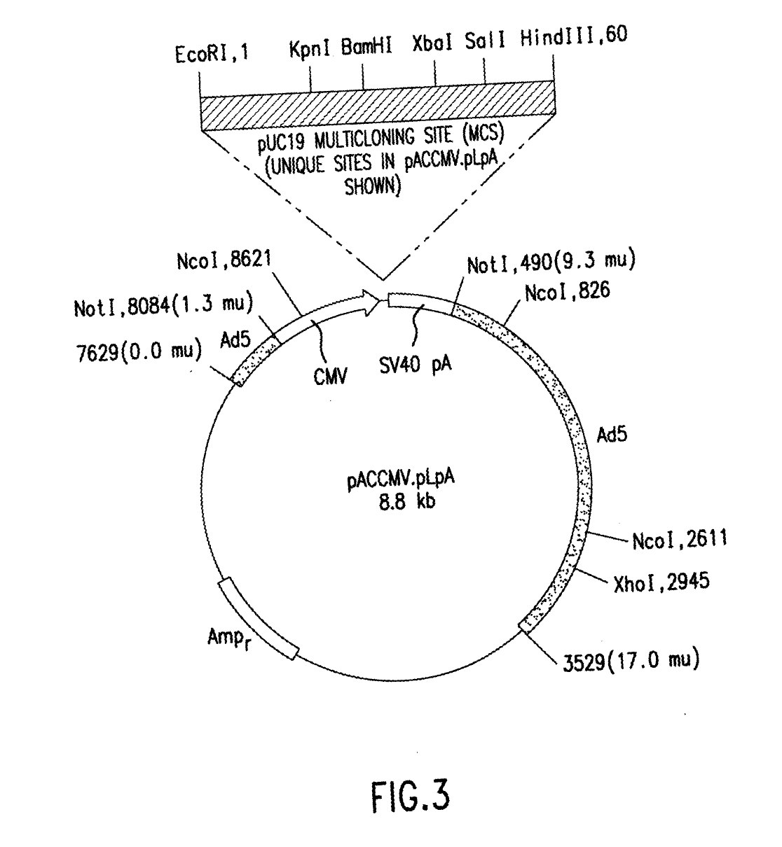

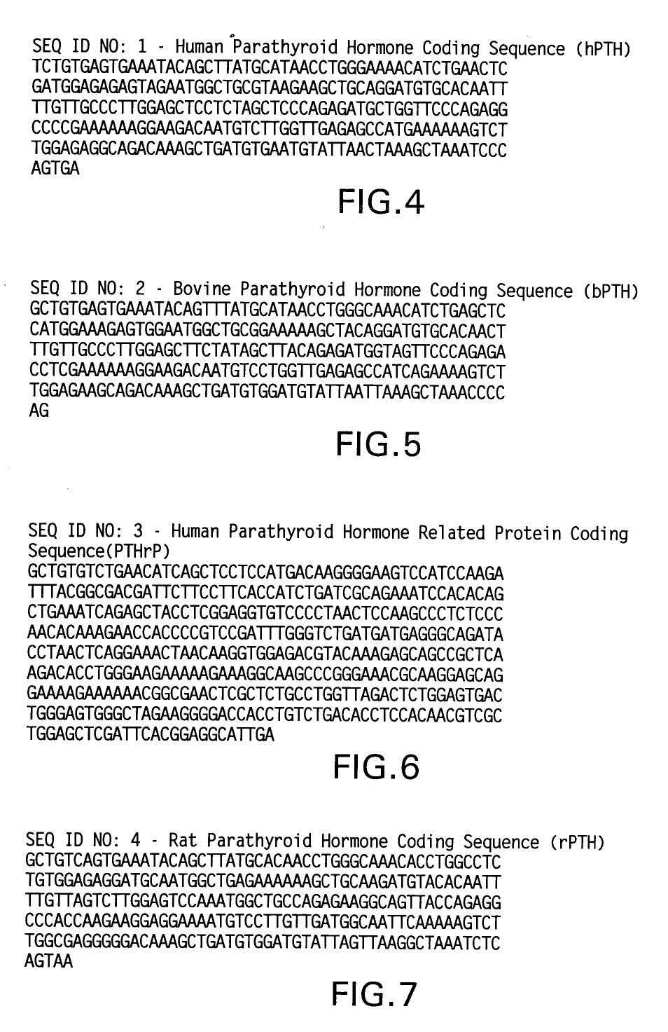

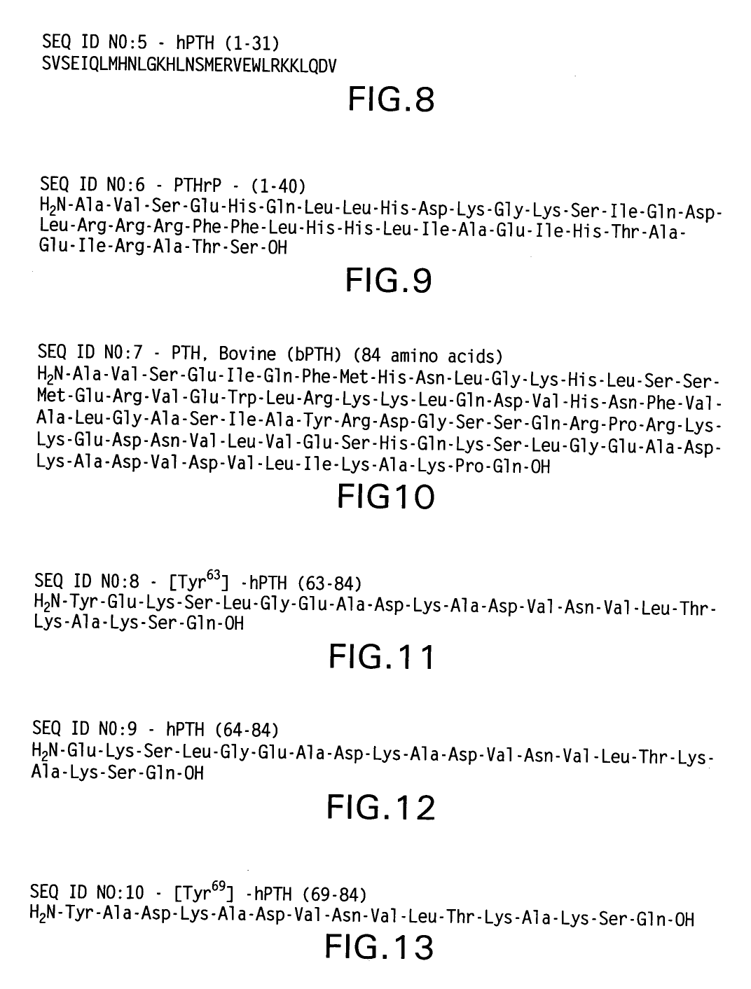

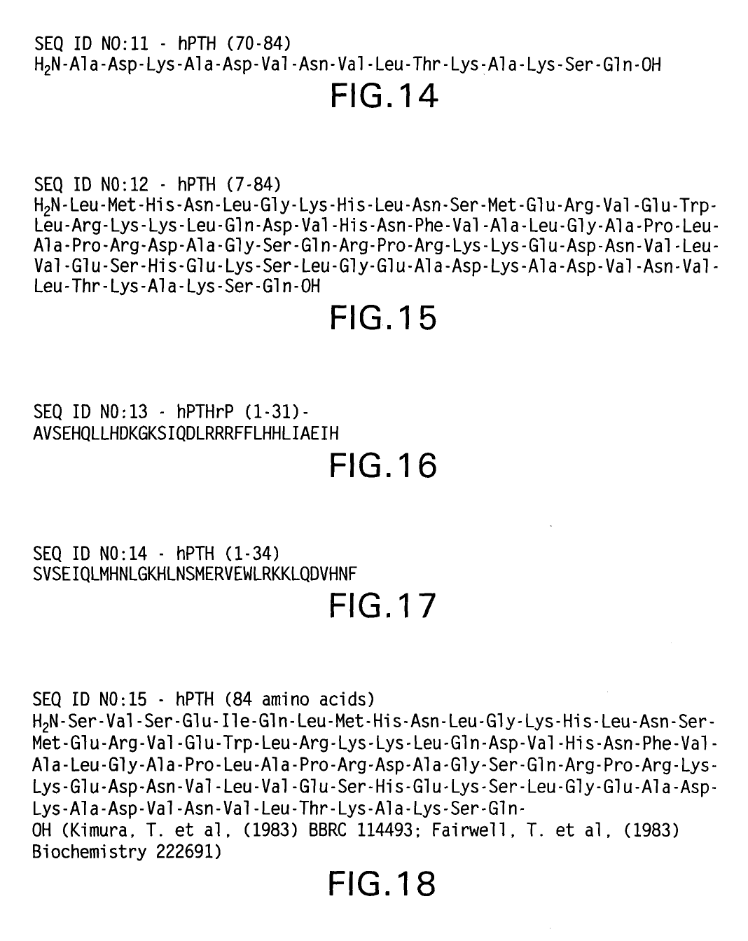

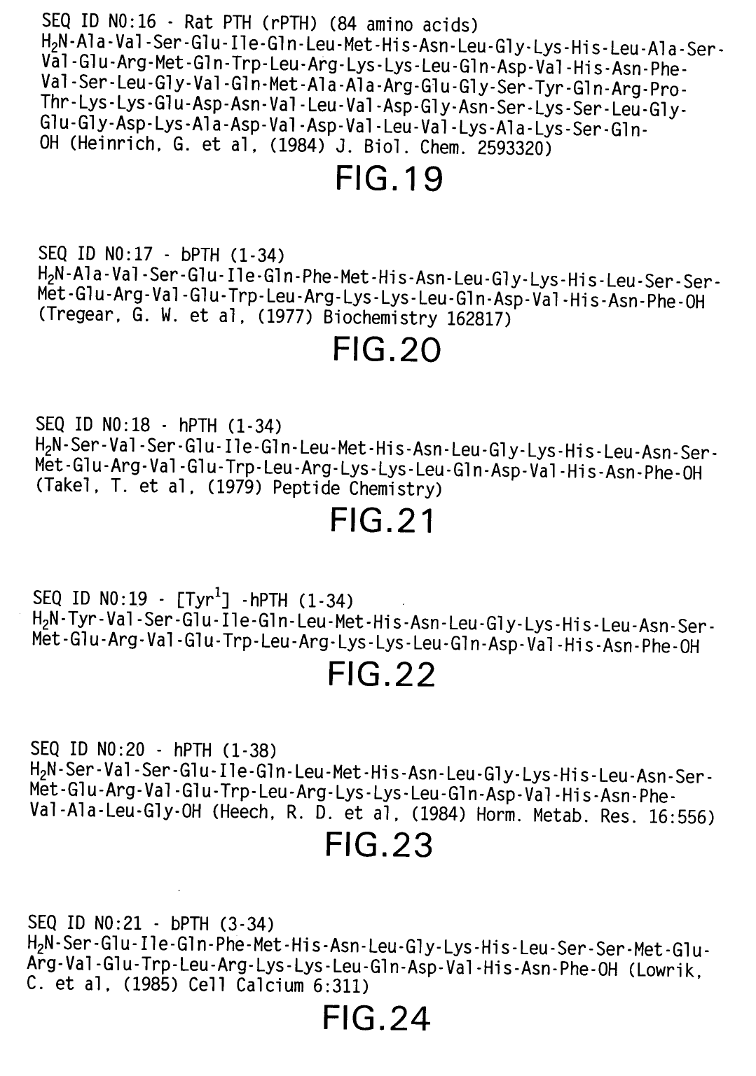

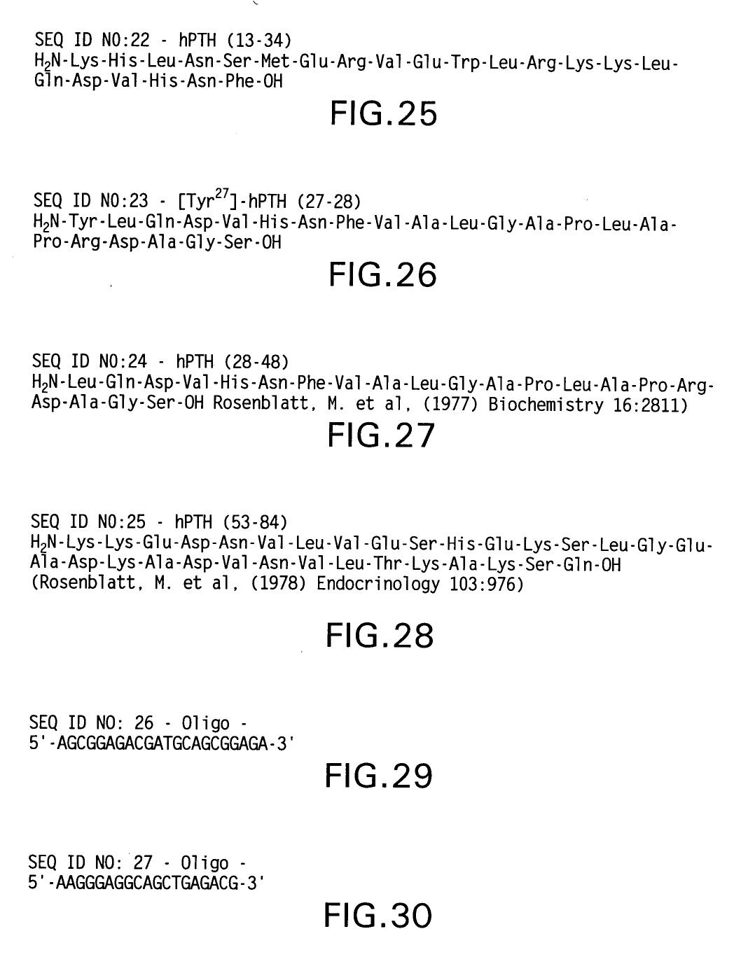

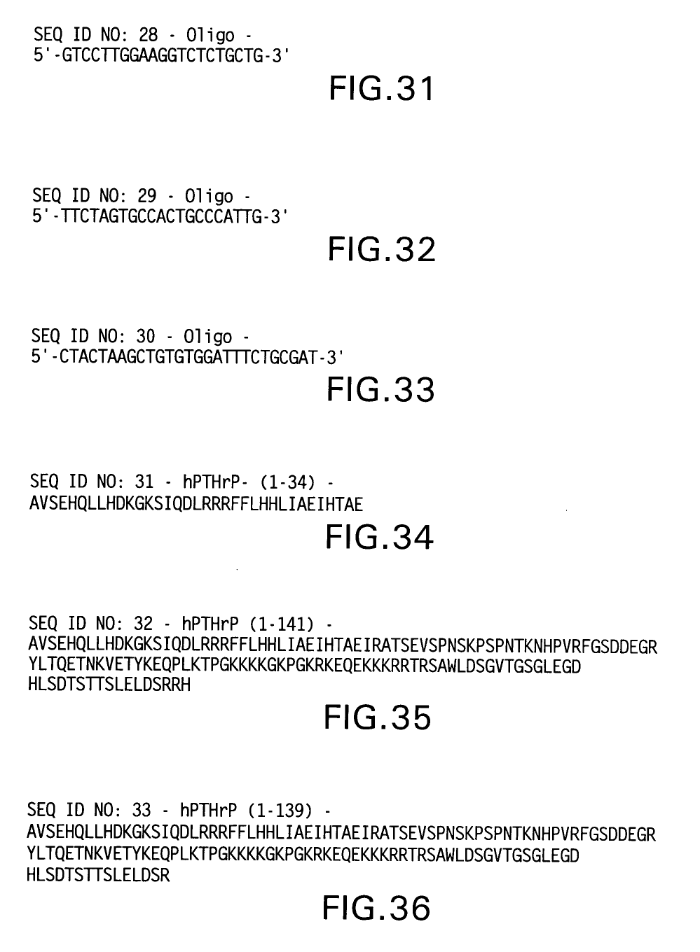

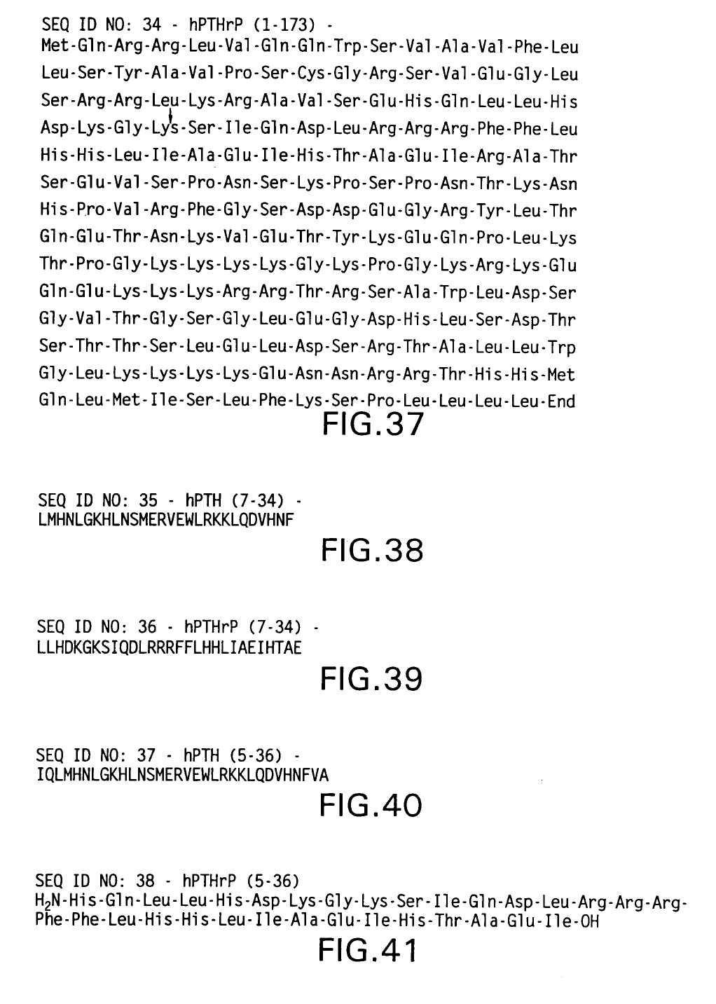

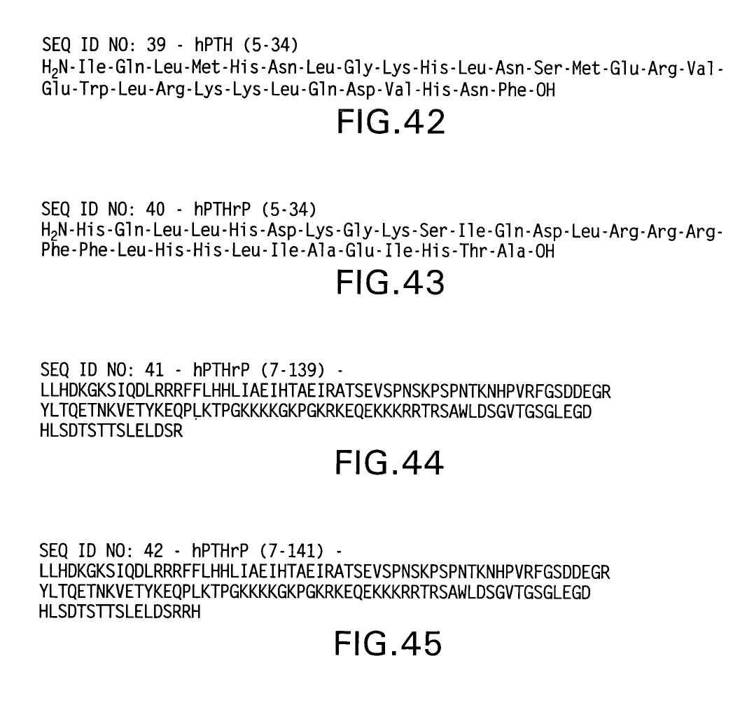

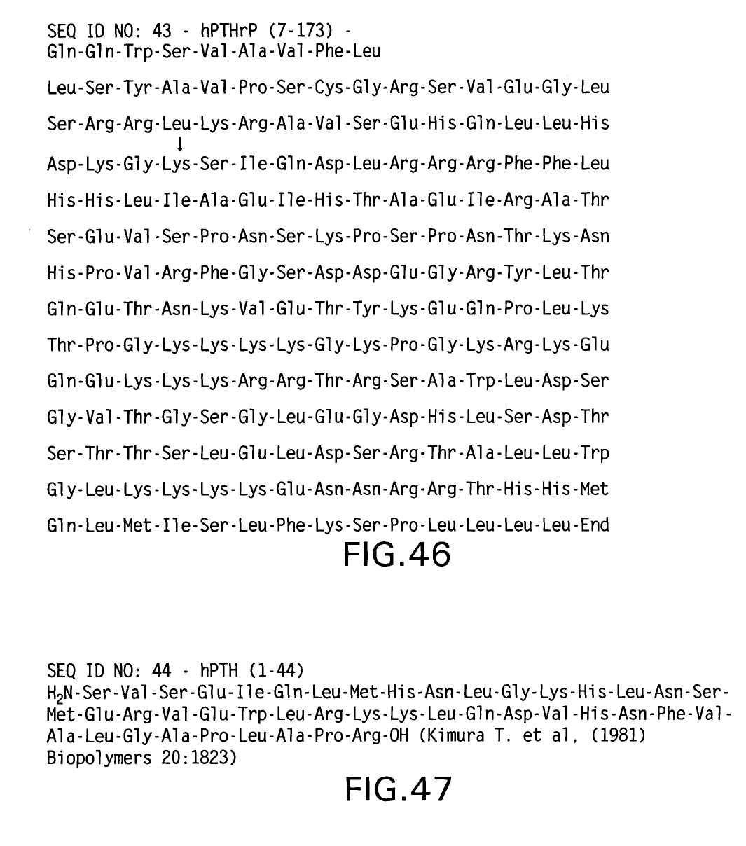

The corresponding mature and fragment forms of PTH or PTHrP cDNAs (FIG. 2) can be subcloned into the adenovirus expression vector, pACCMV.pLpA (FIG. 3). Once the PTH and PTHrP inserts are subcloned and purified they are co-transfected with pJM17 in 293 cells, which contains essential elements of the adenovirus genome to replicate and produce recombinant virions. The virions isolated for the co-transfected 293 cells are infectious but don't have the capacity to replicated in other cell types except 293 cells with the pJM17 vector. The purified pACCMV.pLpA. PTHrP virion particles can then be used for gene transfer of the various PTHrPs cDNAs driven by the CMV promoter in culture and animals (Tomas C. Berker, et al. Methods of Cell Biology, Use of Recombinant Adenovirus for Metabolic Engineering of Mammalian Cells, Vol. 43, Chp 8; pg. 161-187, Academic Press Inc., San Diego, Calif., USA. 1994).

Transfection

Keratinocytes were maintained in MCDB-153 medium. Cells in 24 well dishes at 50%-60% confluence were transfected with 1 μg/ml of PTHrP cDNA which was constructed into pCR3.1 vector (INVITROGEN, San Diego, Calif., USA), empty vector as a control. For each transfection, 0.5 micrograms of DNA and 3 microliters of LIPOFECTAMINE were diluted in 50 microliters of serum free media, respectively, and then combined for a DNA/Liposome complexing incubation for 15 minute at room temperature. DNA/Liposome complex was then incubated on the cells for 3 hours. After 3 hour of transfection, fresh media was added and cells were incubated for 21 hours.

³H-Thymidine Incorporation

³H-thymidine incorporation into DNA was used as an index of cell proliferation as described previously (Smith E. L. et al. J. Invert. Dermatology 86:709 (1986), Holick et al. Proc. Nat. Acad. Sci. U.S.A. 91:8014 (1994)). Twenty-four hours post transfection the medium was replaced with 0.5 ml of fresh basal medium containing [methyl-³H]thymidine (New England Nuclear, Boston, Mass.) and incubated for 3 h at 37° C. ³H-Thymidine incorporation into DNA was stopped by placing the 24-well plates on ice. Unincorporated ³H-thymidine was then removed and the cells were washed three times with ice-cold phosphate-buffered saline. DNA labeled with ³H-thymidine and other macromolecules were first precipitated with ice-cold 5% perchloric acid for 20 min and then extracted with 0.5 ml of 5% perchloric acid at 70 ° C. for 20 min. The radioactivity in the extracts was determined by a liquid scintillation counter. The results were expressed as percent of control.

Experiments have shown that when normal cultured human keratinocytes are transfected with plasmids containing PTHrP (1-141) or PTHrP (1-173) an unexpected enhancement of cell growth is seen, as measured by ³H-thymidine incorporation into epidermal DNA (FIG. 1). These results may be due to proteolysis of the full length peptide.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of inhibiting proliferation or enhancing differentiation of a mammalian skin or hair cell, said method comprising administering to the mammalian skin or hair cell in need of inhibited proliferation or enhanced differentiation with a proliferation-inhibiting or differentiation-enhancing amount of a nucleic acid molecule, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth.

2. The method of claim 1, wherein said nucleic acid molecule is administered as part of a pharmaceutical composition comprising a pharmaceutically acceptable carrier.

3. The method of claim 2, wherein said carrier is a liposome.

4. The method of claim 1, wherein said nucleic acid molecule is contained within a porous biocompatable matrix.

5. The method of claim 1, wherein said peptide encoded by the nucleic acid molecule is PTH (1-34) (SEQ ID NO: 18), PTHrP (1-34) (SEQ ID NO: 31), PTH (1-84) (SEQ ID NO: 15), PTHrP (1-141) ( SEQ ID NO: 32), PTHrP (1-139) (SEQ ID NO: 33) or PTHrP (1-173) (SEQ ID NO: 34).

6. The method of claim 1, wherein said nucleic acid molecule is one of SEQ ID NO: 1, SEQ ID NO: 2, SEQ ID NO: 3, or SEQ ID NO: 4, or a fragment thereof.

7. The method of claim 1, wherein said nucleic acid molecule is administered topically to the mammalian skin or hair cells.

8. The method of claim 1, wherein said method is a method of inhibiting a hyperproliferative skin disorder.

9. The method of claim 8, wherein said hyperproliferative skin disorder is psoriasis, ichthyosis, eczema, acne, actinic keratosis, or skin cancer.

10. The method of claim 1, wherein said method is a method of inhibiting hair growth or preventing hair regrowth.

11. The method of claim 1, wherein said peptide encoded by the nucleic acid molecule has at least 75% sequence identity with the 34 amino acid N-terminal region of hPTH or hPTHrP.

12. The method of claim 1, further comprising administering to the mammalian hair or skin cell an effective amount of an active vitamin D compound.

13. The method of claim 12, wherein said active vitamin D compound is calcipotriene.

14. The method of claim 12, wherein said active vitamin D compound is 1,25-dihydroxyvitamin D₃.

15. The method of claim 12, wherein said active vitamin D compound is 19-nor-1,25-dihydroxyvitamin D₂.

16. The method of claim 12, wherein said active vitamin D compound is 19-nor-1,25-dihyroxyvitamin D₃.

17. The method of claim 12, wherein said nucleic acid molecule and active vitamin D compound are administered topically or parenterally.

18. The method of claim 1, wherein said nucleic acid molecule is operably linked to a promoter.

19. The method of claim 1, wherein said nucleic acid molecule is contained by a plasmid.

20. The method of claim 1, wherein said nucleic acid molecule is contained by a viral vector.

21. A method of inhibiting proliferation or enhancing differentiation of a skin or hair cell of a mammal, said method comprising administering to the mammal in need thereof a proliferation-inhibiting or differentiation-enhancing amount of a nucleic acid molecule and an active vitamin D compound, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth.

22. The method of claim 21, wherein said nucleic acid molecule and said active vitamin D compound are administered as part of a single pharmaceutical composition.

23. The method of claim 21, wherein said nucleic acid molecule and said active vitamin D compound are administered as part of separate pharmaceutical compositions.

24. The method of claim 21, wherein said nucleic acid molecule is administered parentally.

25. The method of claim 21, wherein said active vitamin D compound is administered topically.

26. The method of claim 21, wherein said active vitamin D compound is administered orally.

27. The method of claim 21, wherein said nucleic acid molecule is encapsulated within a liposome.

28. The method of claim 21, wherein said nucleic acid molecule is contained within a porous biocompatable matrix.

29. A method of inducing proliferation of a mammalian skin or hair cell, said method comprising administering to the mammalian skin or hair cell in need of proliferation with a proliferation-inducing amount of a nucleic acid molecule, wherein the peptide encoded by the nucleic acid molecule is at least 3 amino acids long, has at least 10% sequence identity with the 34 amino acid N-terminal region of hPTH or hPTHrP, and, when expressed, is capable of blocking the inhibition of proliferation or stimulation of differentiation in vitro of cultured human keratinocytes by PTH (1-34), 1,25(OH)₂D₃ or PTHrP (1-34), or in vivo in mouse skin by stimulating skin cell proliferation or accelerating hair cycle progression or stimulating hair cell growth.

30. The method of claim 29, wherein said nucleic acid molecule is administered as part of a pharmaceutical composition comprising a pharmaceutically acceptable carrier.

31. The method of claim 30, wherein said carrier is a liposome.

32. The method of claim 29, wherein said nucleic acid molecule is contained within a porous biocompatable matrix.

33. The method of claim 29, which is a method of stimulating skin cell growth, rejuvenating aged skin, preventing skin wrinkles, treating skin wrinkles, enhancing wound healing, stimulating hair growth, maintaining hair growth, treating or preventing female or male pattern baldness, or treating chemotherapy induced alopecia.

34. The method of claim 29, which is a method of stimulating epidermal cell growth or hair follicle cell growth.

35. The method of claim 29, wherein said peptide encoded by the nucleic acid molecule is PTH (7-34) (SEQ ID NO: 35), PTHrP (7-34) (SEQ ID NO: 36), PTH (5-36) (SEQ ID NO: 37), PTHrP (5-36) (SEQ ID NO: 38), PTH (5-34) (SEQ ID NO: 39), PTHrP (5-34) (SEQ ID NO: 40), PTH (7-84) (SEQ ID NO: 12), PTHrP (7-139) (SEQ ID NO: 41), PTHrP (4-141) (SEQ ID NO: 42), or PTHrP (7-173) (SEQ ID NO: 43)

36. The method of claim 1, wherein said nucleic acid molecule is one of SEQ ID NO: 1, SEQ ID NO: 2, SEQ ID NO: 3, or SEQ ID NO: 4, or a fragment thereof.

37. A composition comprising a proliferation-inhibiting or differentiation-enhancing amount of a nucleic acid molecule encapsulated within a liposome, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth.

38. The method of claim 37, wherein said nucleic acid molecule is contained within a porous biocompatable matrix.

39. A composition comprising a proliferation-inducing amount of a nucleic acid molecule encapsulated within a liposome, wherein the peptide encoded by the nucleic acid molecule is at least 3 amino acids long, has at least 10% sequence identity with the 34 amino acid N-terminal region of hPTH or hPTHrP, and, when expressed, is capable of blocking the inhibition of proliferation or stimulation of differentiation in vitro of cultured human keratinocytes by PTH (1-34), 1,25(OH)₂D₃ or PTHrP (1-34), or in vivo in mouse skin by stimulating skin cell proliferation or accelerating hair cycle progression or stimulating hair cell growth.

40. A composition comprising a proliferation-inhibiting or differentiation-enhancing amount of a nucleic acid molecule and an active vitamin D compound, wherein the peptide encoded by the nucleic acid molecule is at least 3 amino acids long, has at least 10% sequence identity with the 34 amino acid N-terminal region of hPTH or hPTHrP, and, when expressed, is capable of inhibiting proliferation or enhancing differentiation in vitro of cultured human keratinocytes, or in vivo in mouse skin by inhibiting skin cell proliferation or hair cycle progression or hair cell growth.

41. The composition of claim 40, wherein at least one of said nucleic acid molecules or active vitamin D compound is encapsulated by liposomes.
